(19) United States
(12) Patent Application Publication
(10) Pub. No.: US 2005/0287561 A1
(43) Pub. Date: Dec. 29, 2005

(54) Genomic proxy microarrays to identify microbial quantitative trait loci

(75) Inventors: Harrison Fields Dillon, Palo Alto, CA (US); Jonathan Seth Wolfson, Palo Alto, CA (US)

Correspondence Address: SOLAZYME, INC., 3475 - T Edison Way, Menlo Park, CA, 94025, US

(73) Assignee: Solazyme Inc., Menlo Park, CA (US)

(21) Appl. No.: 11/124,708

(22) Filed: May 9, 2005

Related U.S. Application Data
(60) Provisional application No. 60/569,765, filed on May 10, 2004.

(51) Int. Cl. C12Q 1/68; C12N 15/74

(52) U.S. Cl. 435/6; 435/471

(57) ABSTRACT

Methods are provided for engineering microbial organisms to perform a desired function at higher levels than naturally existing strains. The diversity within and between species of the level of (a) genomic diversity and (b) performance of the desired function are used to identify genes that can be optimized for increasing the performance of the desired function

### BACKGROUND OF THE INVENTION

Identifying genes that can cause an increase in a desirable function of an organism is a desirable goal. Typical methods include random disruption of genes followed by screening of organisms to identify transformants that can no longer perform the desired function. The genomic location of the random disruption event is mapped and the gene is identified and studied. These methods are not capable of identifying genes that improve the desired function when expressed at a higher level but are not absolutely necessary for the performance of the desired function.

### BRIEF SUMMARY OF THE INVENTION

The methods provided herein are useful for identifying genes that enhance a desired function. The methods are also useful in enhancing a desired function through the expression of genes identified by other methods of the invention. Methods are also provided for further enhancing a desired function by inducing nucleic acid exchange between two or more independent transformants that each performs a desired function to generate progeny that perform the desired trait better than either parent.

Genomic proxy microarrays are generated corresponding to a single set of protein sequences (which can be 1, 10, 100 1,000, 10,000 or more proteins sequences) that contain immobilized oligonucleotides that encode the set of proteins in a particular codon usage regime. Cells are tested for a desired trait, which is measured, and thereafter mRNA samples are taken from the cells. The mRNA samples are turned into labeled cDNA samples that are preferably fragmented. The cDNA fragments are then applied to the microarray(s). Samples are hybridized to microarrays that contain the set of protein sequences encoded in the preferred codon usage regime of the cell from which the sample was generated. The expression level of each gene of the microarrays is measured. The expression level of each gene identified from one sample is then compared to the level of expression of the gene in other organisms, from other samples. The expression level of each gene is correlated with the level at which the desired trait is performed by each strain tested. Genes that show a higher level of expression in cells that perform the trait at higher levels compared to genes that show a lower level of expression and a lower level of performance of the desired function are opportune targets for upregulation to create new strains that perform the desired trait at a higher level than without expression of the opportune targets. Two or more new strains expressing different opportune targets, wherein each new strain performs the desired trait at a higher level than the strain it was derived from before transformation with the opportune target expression vector, are then induced to undergo nucleic acid exchange to produce progeny that perform the desired trait at an even higher level than any individual parental strain.

Some methods involve culturing two or more genomically diverse microorganisms under conditions in which at least two genomically diverse microorganisms perform a desired function; measuring the level of performance by the at least two genomically diverse microorganisms of the desired function; isolating mRNA from the at least two genomically diverse microorganisms that perform the desired function at different levels; hybridizing the mRNA or a nucleic acid derivative thereof to a microarray containing one or more immobilized cDNA sequences; and identifying one or more opportune targets that are expressed at a higher level in a microorganism that performs the desired function at a higher level compared to the expression level of the opportune target in a different microorganism that performs the desired function at a lower level. Some methods further comprise expressing the one or more opportune targets in a transformed test strain using a heterologous promoter other than the natural promoter(s) of the one or more opportune targets; and screening or selecting for an increase in the level of performance of the desired function in the transformed test strain compared to a nontransformed test strain. Some methods further comprise identifying a transformed test strain that exhibits an increase in the desired function, including wherein at least two independent transformed test strains expressing different opportune targets are identified. Some methods are performed, further comprising placing the at least two independent transformed test strains are placed in conditions where they undergo nucleic acid exchange; and screening or selecting progeny cells for a further increase in the desired function at a level higher than that exhibited by at least one of the at least two independent transformed test strains. In a further embodiment the progeny cells are screened or selected for a further increase in the desired function at a level higher than that exhibited by all of the at least two independent transformed test strains. A further embodiment comprises a first progeny cell that exhibits a further increase in the desired function at a level higher than that exhibited by all of the at least two independent transformed test strains is placed in conditions where it undergoes nucleic acid exchange with a second distinct progeny cell that also exhibits a further increase in the desired function at a level higher than that exhibited by all of the at least two independent transformed test strains to produce additional progeny; and screening or selecting the additional progeny for performance of the desired function at a level higher than that exhibited by at least one of the first or second progeny cells. In a further embodiment the additional progeny are screened or selected for performance of the desired function at a level higher than that exhibited by the first and second independent progeny cells.

In some methods the desired function is hydrogen production, carbon sequestration, astaxanthin production dissolved solid transport (such as Na⁺ or Cl⁻), or degradation or chelation of an environmental toxin. For hydrogen production an assay can be screened using a multiwell plate of independent genomically diverse microorganisms in liquid culture media, and an increase in hydrogen production is identified by a change in optical properties of a chemochromic film placed on top of the plate.

In some methods the genomically diverse microorganisms are listed in Tables 1, 2 or 3. In some method, two or more genomically diverse microorganisms are generated by inducing genomic diversity through mutagenesis of cells, such as cells of strains listed in Tables 1, 2 or 3, or are microorganisms derived from a microorganism listed in Tables 1, 2 or 3.

In some methods a plurality of distinct microarrays are used, each microarray containing nucleic acid sequences that encode the same set of protein sequences but wherein at least two distinct microarrays from the plurality encode the protein sequences using different codon usage regimes. In some methods the codon usage regimes include at least two regimes selected from the list consisting of those of Chlamydomonas reinhardtii, Chlamydomonas culleus, Chlamydomonas debaryana, Chlamydomonas dorsoventralis, Chlamydomonas hydra, Chlamydomonas moewusii, Chlamydomonas noctigama, Chlamydomonas eugamentos, and Chlamydomonas incerta.

Nucleic acid exchange in some methods can be sexual recombination, bacterial conjugation, virus-mediated or protoplast fusion.

In some methods at least two independent transformed test strains are green algae and sexual recombination is induced by removing nitrogen from the culture media as described and referenced in U.S. patent application Ser. No. 10/763,712.

In some methods distinct culture conditions are used to induce cells to perform the same desired function. In some methods the distinct conditions include depriving the cells of sulfur in continuous light; and placing cells under anaerobic conditions in the dark followed by exposure to light, wherein the cells are green algae; and the desired function is hydrogen production.

In some methods a heterologous promoter in operable linkage with the opportune target is activated by light. In some methods the same heterologous promoter drives expression of all opportune targets.

In some methods at least 40 or at least 200 genomically diverse independent strains of microorganisms of a species are analyzed. In some methods at least 2 genomically diverse independent strains of microorganisms from each of at least 2 distinct species are analyzed. In some methods at least 200 genomically diverse independent strains of microorganisms from each of at least 5 distinct species are analyzed.

In some methods chemical mutagenesis is performed to induce single nucleotide polymorphisms to generate genomically diverse microorganisms. In some methods mutagenesis is performed by random insertion of one or more promoters into the genomes of genomically diverse microorganisms or genomically identical microorganisms. In some methods the promoters are identical. In other methods the promoters are not identical. In some methods at least two genomically diverse microorganisms are genomically diverse only from naturally occurring diversity and not induced genomic diversity.

### DETAILED DESCRIPTION OF THE INVENTION

U.S. patent application Ser. Nos. 10/411,910, 10/287,750, 60/500,032 and 10/763,712 are incorporated by reference for all purposes. This application claims priority to U.S. Patent Application No. 60/569,765, filed May 10, 2004.

I Introduction

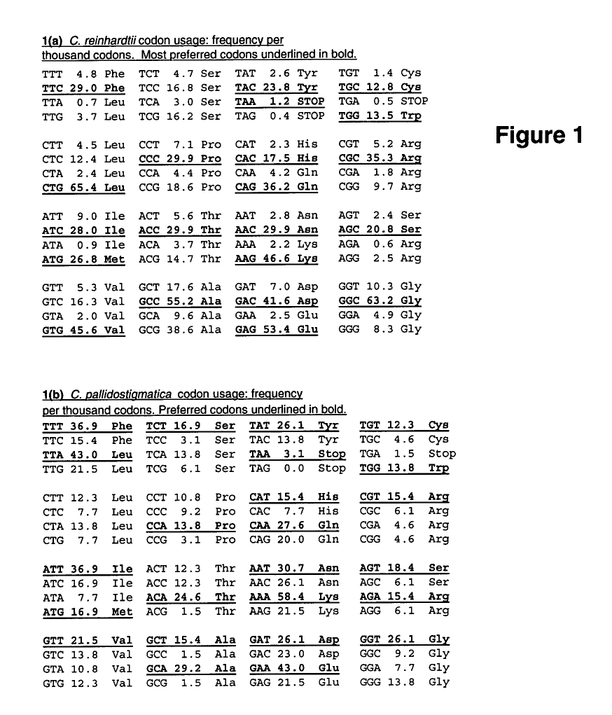

It has long been known that different organisms have different codon usage regimes. C. reinhardtii, for example, has a stringent codon usage regime. It is frequently not possible to express a foreign gene in C. reinhardtii without constructing a synthetic gene that uses codons preferred in C. reinhardtii. Other species of Chlamydomonas, such as C. pallidostigmatica, possess a completely different codon usage regime (see FIGS. 1a-b). As a result, different species of Chlamydomonas possess genomes that have many genes that have significant sequence identity at the amino acid level but are completely divergent at the nucleotide level. In many cases protein sequences are conserved between species yet the corresponding cDNA sequences of these proteins possess no more nucleotide similarity to each other than random sequence.

Because different species within a genus possess different metabolic capabilities, it is useful to examine genome-wide expression patterns of numerous species of microbes performing a common metabolic function with varying levels of productivity.

A large number of distinct strains of organisms of two or more species that can perform a desired function are quantitatively tested for that function.

Strains from each species that perform the desired function at the highest level and strains from each species that perform the desired function at the lowest level are selected for expression analysis on microarrays. For example, if 200 strains of a species are used, the top 20% (40 strains) and the bottom 20% (40 strains) are analyzed. Preferably, multiple species are analyzed (such as 8), with numerous strains in each species. For a 10,000 gene microarray, this example yields quantitative data for expression of 10,000 genes in 80 strains of 8 species to produce 6,400,000 data points that are correlated with performance of the desired trait.

Genes that are consistently expressed at higher levels in strains that perform the desired function at the highest levels than in strains that perform the desired function at lowest levels are then expressed as cDNAs in transformed test strains. Increases in performance of the desired trait are assayed with a non-transformed test strain as a control. A strain that exhibits an increase in the desired trait when expressing an opportune target is induced to undergo nucleic acid exchange with one or more independent strains that express different opportune targets and also exhibit an increase in performance of the desired trait to produce further improved progeny that inherit both opportune target expression vectors. Multiple rounds of nucleic acid exchange using improved strains (containing a validated opportune target) creates strains that contains a large number of validated opportune targets that individually and together increase the capacity of progeny cells to perform a desired function.

As an example, Eight Chlamydomonas species have been demonstrated to photoproduce different levels of hydrogen (H₂): C. reinhardtii, C. moewusii, C. chlamydogama, C. culleus, C. debaryana, C. dorsoventralis, C. hydra, and C. noctigama (Brand et al. Biotech. Bioeng. 33:1482-8 (1989)). It is known that different species of Chlamydomonas and different strains of the same species photoproduce different levels of H₂. For example, it has also been demonstrated that most strains of C. moewusii photoproduce more H₂ gas than C. reinhardtii (Greenbaum, Biophys. J. 54:365-368 (1988)). In addition, the same research has demonstrated that different Chlamydomonas strains of the same species produce different levels of H₂. Natural genetic variation in green algae causes this differential metabolism. Specifically, these intra- and inter-species differences in H₂ production are due to genomic SNP variation and gene regulation differences. For example, a high level of SNP divergence has been demonstrated for two C. reinhardtii strains: the 137C strain, isolated in Massachusetts, and the S1D2 strain, isolated in Minnesota (Vysotskaia et al., Plant Physiol. 127(2):386-9 (2001)). These differences in H₂ production capability and genomic sequence are used to identify opportune targets that are expressed to create highly productive Chlamydomonas strains.

The following are strains of C. reinhardtii are generally genomically diverse: (strain numbers of the Chlamydomonas Genetics Center, Duke University): CC-124, CC-125, CC-1690, CC-1692, CC-407, CC-408, CC-1952, CC-2290, CC-2342, CC-2343, CC-2344, CC-2931, CC-2932, CC-2935, CC-2936, CC-2937, CC-2938, CC-2935, CC-2936, CC-2937, CC-2938, CC-3059, CC-3060, CC-3061, CC-3062, CC-3063, CC-3064, CC-3065, CC-3067, CC-3068, CC-3071, CC-3073, CC-3074, CC-3075, CC-3076, CC-3078, CC-3079, CC-3080, CC-3082, CC-3083, CC-3084, CC-3086, CC-1373 and CC-3087. These strains were isolated from geographically diverse regions and contain SNPs relative to each other's genome. The interspecies and intraspecies differences in H₂ production levels of these organisms are used to identify genes responsible for H₂ production.

A nucleic acid is operably linked when it is placed into a functional relationship with another nucleic acid sequence. For instance, a promoter or enhancer is operably linked to a coding sequence if it increases the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term naturally-occurring is used to describe an object that can be found in nature as distinct from being artificially produced by man.

Screening is, in general, a two-step process in which one first determines which cells do and do not express a screening marker and then physically separates the cells having the desired property. Selection is a form of screening in which identification and physical separation are achieved simultaneously by expression of a selection marker, which, in some genetic circumstances, allows cells expressing the marker to survive while other cells die (or vice versa). Selection markers include drug and toxin resistance genes. Although spontaneous selection can and does occur in the course of natural evolution, in the present methods selection is performed by man.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “significant sequence identity” as used herein denotes a characteristic of a polynucleotide or polypeptide sequence, wherein the polynucleotide or polypeptide comprises a sequence that has at least 50 percent sequence identity, preferably at least 65 percent identity and often 70 to 95 percent sequence identity, more, usually at least 70 percent sequence identity as compared to a reference sequence over a comparison window of at least 20 nucleotide or amino acid positions, frequently over a window of at least 25-50 nucleotides or amino acids, wherein the percentage of sequence identity is calculated by comparing the reference sequence to the polynucleotide sequence which may include deletions or additions which total 20 percent or less of the reference sequence over the window of comparison. Sequence comparison is typically performed using the BLAST or BLAST 2.0 algorithm with default parameters.

I Examples of Desired Functions Traits that can be Optimized

Any desired function can be optimized using the methods provided herein. Hydrogen production: The ability to produce hydrogen is a desirable function because hydrogen gas is used for a variety of industrial purposes including oil refining, electricity generation, as a direct transportation fuel, fiber optic cable production, and other uses. H₂ may be detected using a variety of methods such chemochromic sensing films that contain transition metals (see U.S. Pat. No. 6,277,589). Such films change from clear to dark grey-blue when exposed to H₂, and when placed in proximity to cells that produce different amounts of H₂ they identify cells that produce more H₂ than others. There are other methods, both direct and indirect, that are used to detect hydrogen, such as spectroscopic methods (see U.S. Pat. Nos. 5,100,781 and 6,309,604). Other types of gas sensors and films suitable for detection of hydrogen are known in the art. See U.S. Pat. Nos. 5,100,781, 6,484,563, 6,265,222 and 6,006,582. Gas chromatography can also be used.

Astaxanthin Production:

Production of astaxanthin and other nutritional supplements are desirable functions. These functions can be assayed for using techniques such as mass spectrometry (Takaichi,S., Matsui,K., Nakamura,M., Muramatsu,M. and Hanada,S. Fatty acids of astaxanthin esters in krill determined by mild mass spectrometry. Comp. Biochem. Physiol. B, 136, 317-322 (2003); Haematococcus pluvialis UTEX 16, Choi et al., Biotechnol Prog. 2002 Nov-Dec;18(6):1170-5.). Other fatty acid molecules are assayed using mass spectrometry (Blokker,P., Pel,R., Akoto,L., Brinkman,U. A. T. and Vreuls,R. J. J. At-line gas chromatographic-mass spectrometric analysis of fatty acid profiles of green microalgae using a direct thermal desorption interface. J. Chromatogr. A, 959, 191-201 (2002); Viron,C., Saunois,A., Andre,P., Perly,B. and Lafosse,M. Isolation and identification of unsaturated fatty acid methyl esters from marine micro-algae. Anal. Chim. Acta, 409, 257-266 (2000)).

Dissolved Solid Transport

Cells are assayed for the ability to transport dissolved solids such as NaCl. Cells are tested for the ability to transport the solids into our out of the cell. For example, Duniella salina cells are tested for the ability to transport labeled sodium (such as Na²²) into or out of the cell. D. salina, a seawater algae, maintains an intracellular salt concentration significantly lower than the surrounding seawater. Strains that have improved salt transport capabilities are assayed for. Sodium and chloride transport assays are known in the art (Kidney Int. 1997 July;52(1):229-39; Kidney Int. 2004 May;65(5):1676-83; Proc Natl Acad Sci USA. 2004 Feb. 17;101(7):2064-9).

Ethanol Production

Production of ethanol as a fuel by eukaryotic or prokaryotic cells is a desirable trait (see Appl Biochem Biotechnol. 2003 Spring;105-108:87-100; Appl Microbiol Biotechnol. 2003 December;63(3):258-66.). Methods of assaying for ethanol are well known. Virtually any molecule can be assayed using mass spectrometry.

Bioremediation:

The ability to degrade or chelate environmental toxins is a desirable trait. These capabilities are assayed using known methods (Ahmann, D., L. R. Krumholz, H. F. Hemond, D. R. Lovley, and F. M. M. Morel (1997) Microbial mobilization of arsenic from sediments of the Aberjona Watershed. Environ. Sci. Technol. 31:2923-2930; Newman, D. K., Kennedy, E. K., Coates, J. D., Ahmann, D., Ellis, D. J., Lovley, D. R., and Morel, F. M. M. (1997) Dissimilatory arsenate and sulfate reduction in Desulfotomaculum auripigmentum sp. nov. Archives of Microbiology 168:380-388; Ahmann, D., A. L. Roberts, L. R. Krumholz, and F. M. M. Morel (1994) Microbe grows by reducing arsenic. Nature 351:750)

Carbon Sequestration:

The ability to sequenster carbon from gaseous CO₂ is a desirable trait. C¹⁴ can be used as a labeled substrate. For example, green algae are assayed for an enhanced ability to retain C¹⁴ after exposure to labeled CO₂ gas. See U.S. Patent Application 20030073135 for other examples.

II Strains and Microorganisms

Any single celled microbe can be used in the methods described herein. Examples include green algae such as Chlamydomonas and Scenedesmus, yeast, E. coli, and other organisms.

TABLE 1
Azotobacter vinelandii AvOP
Chlorobium tepidum
Chloroflexus aurantiacus J-10-fl
Nitrosomonas europaea ATCC25978
Nostoc punctiforme ATCC29133
Prochlorococcus marinus MED4
Prochlorococcus marinus MIT9313
Rhodopseudomonas palustris CGA009
Rhodospirillum rubrum ATCC11170
Synechococcus WH8102
Thalassiosira pseudonana
Trichodesmium erythraeum IMS101
Methanobacterium thermoautotrophicum Delta H
Methanococcoides burtonii DSM6242
Methanococcus jannaschii DSM2661
Methanosarcina barkeri Fusaro
Acidithiobacillus ferrooxidans
Burkholderia LB400 (degradesPCBs)
Caulobacter crescentus
Dechloromonas RCB
Dehalococcoides ethenogenes
Deinococcus radiodurans R1
Desulfitobacterium hafniense DCB-2
Desulfovibrio desulfuricans G20
Desulfovibrio vulgaris (H₂ producer)
Desulfuromonas acetoxidans
Ferroplasma acidarmanus fer1
Geobacter metallireducens
Geobacter sulfurreducens
Mesorhizobium BNC1
Methylococcus capsulatus
Novosphingobium aromaticivorans F199
Pseudomonas fluorescens PFO-1
Pseudomonas putida
Ralstonia metallidurans CH34
Rhodobacter sphaeroides 2.4.1
Shewanella oneidensis MR-1
Clostridium thermocellum ATCC27405
Cytophaga hutchinsonii
Microbulbifer 2-40
Phanerochaete chrysosporium
Thermobifida fusca YX
Aquifex aeolicus VF5
Archaeoglobus fulgidus DSM4304
Bifidobacterium longum DJO10A
Brevibacterium linens BL2
Clostridium acetobutylicum (Produces acetone, butanol, and ethanol);
Ehrlichia chaffeensis Sapulpa
Ehrlichia canis Jake
Halobacterium halobium plasmid
Lactobacillus brevis ATCC367
Lactobacillus bulgaricus ATCCBAA-365
Lactobacillus casei ATCC334
Lactobacillus gasseri ATCC33323
Lactococcus lactis cremoris SK11
Leuconostoc mesenteroides
Magnetococcus MC-1
Magnetospirillum magnetotacticum MS-1 ATCC31632
Oenococcus oeni PSU1
Pediococcus pentosaceus ATCC25745
Pseudomonas syringae B728a
Pyrobaculum aerophilum
Pyrococcus furiosus
Streptococcus thermophilus LMD-9
Thermotoga maritima M5B8
Borrelia burgdorferi B31
Brucella melitensis 16M
Enterococcus faecium
Exiguobacterium 255-15
Haemophilus somnus 129PT
Mycoplasma genitalium G-37
Psychrobacter 273-4
Streptococcus suis 1591
Xylella fastidiosa Dixon
Xylella fastidiosa

TABLE 2
Chlamydomonas species and strains: (numbers are accession
numbers from the UTEX collection, http://www.bio.utexas.edu/research/
utex/class/class.html)

102 Chlamydomonas Chlamydogama
1060 Chlamydomonas culleus Ettl
1344 Chlamydomonas debaryana var. cristata Ettl
228 Chlamydomonas dorsoventralis Pascher
4 Chlamydomonas hydra Ettl
9 Chlamydomonas moewusii Gerloff
10 Chlamydomonas moewusii Gerloff
91 Chlamydomonas moewusii Gerloff
92 Chlamydomonas moewusii Gerloff
94 Chlamydomonas moewusii Gerloff
96 Chlamydomonas moewusii Gerloff
97 Chlamydomonas moewusii Gerloff
223 Chlamydomonas moewusii Gerloff
694 Chlamydomonas moewusii Gerloff
695 Chlamydomonas moewusii Gerloff
697 Chlamydomonas moewusii Gerloff
699 Chlamydomonas moewusii Gerloff
701 Chlamydomonas moewusii Gerloff
703 Chlamydomonas moewusii Gerloff
704 Chlamydomonas moewusii Gerloff
705 Chlamydomonas moewusii Gerloff
707 Chlamydomonas moewusii Gerloff
709 Chlamydomonas moewusii Gerloff
711 Chlamydomonas moewusii Gerloff
713 Chlamydomonas moewusii Gerloff
715 Chlamydomonas moewusii Gerloff
716 Chlamydomonas moewusii Gerloff
751 Chlamydomonas moewusii Gerloff
812 Chlamydomonas moewusii Gerloff
2018 Chlamydomonas moewusii Gerloff
2019 Chlamydomonas moewusii Gerloff
2275 Chlamydomonas moewusii Gerloff
2276 Chlamydomonas moewusii Gerloff
1053 Chlamydomonas moewusii var. microstigmata
1054 Chlamydomonas moewusii var. microstigmata (Lund) Ettl
576 Chlamydomonas moewusii var. rotunda Tsubo
577 Chlamydomonas moewusii var. rotunda Tsubo
2602 Chlamydomonas moewusii var. rotunda Tsubo:
2603 Chlamydomonas moewusii var. rotunda Tsubo:
1033 Chlamydomonas moewusii var. tenuichloris Tsubo
1034 Chlamydomonas moewusii var. tenuichloris Tsubo
1338 Chlamydomonas noctigama Korsh.
89 Chlamydomomas reinhardtii Dang., mating type minus
90 Chlamydomonas reinhardtii Dang.,
2247 Chlamydomonas reinhardtii Dang.
2337 Chlamydomonas reinhardtii Dang.
LB 2607 Chlamydomonas reinhardtii Dang.:
LB 2608 Chlamydomonas reinhardtii Dang.:
LB 796 Chlamydomonas sp.
LB 1028 Chlamydomonas sp.
2440 Chlamydomonas sp.
Also incorporated by reference are all strains listed in Table II of Brand et al. Biotech. Bioeng. 33: 1482-8 (1989)

TABLE 3
Chlamydomonas strains: (numbers are accession numbers from the
Chlamydomonas Genetics Center at Duke University
http://www.biology.duke.edu/chlamy_genome/index.html)

CC-124
CC-125
CC-1690
CC-1692
CC-407
CC-408
CC-1952
CC-2290
CC-2342
CC-2343
CC-2344
CC-2931
CC-2932
CC-2935
CC-2936
CC-2937
CC-2938
CC-2935
CC-2936
CC-2937
CC-2938
CC-3059
CC-3060
CC-3061
CC-3062
CC-3063
CC-3064
CC-3065
CC-3067
CC-3068
CC-3071
CC-3073
CC-3074
CC-3075
CC-3076
CC-3078
CC-3079
CC-3080
CC-3082
CC-3083
CC-3084
CC-3086
CC-1373
CC-3087

Preferred organisms are capable of nucleic acid exchange methods such as sexual recombination (mating), conjugation, viral infection, and other methods. Examples of nucleic acid exchange can be found in U.S. Pat. Nos. 6,716,631; 6,528,311; 6,379,964; 6,352,859; 6,335,198; 6,326,204; 6,287,862; 6,251,674.

III Genomic Diversity

Naturally Occurring Genomically Diverse Microorganisms

Genomic diversity naturally exists within microbes, even from the same species, in independent isolates. For example, it has been demonstrated that some strains of Chlamydomonas reinhardtii have a high level of genomic diversity at the Single Nucleotide Polymorphism (SNP) level (Vysotskaia et al., Plant Physiol. 127(2):386-9 (2001). Many of the strains listed in tables 2 and 3 are genomically diverse within a single species.

Induced Genomic Diversity

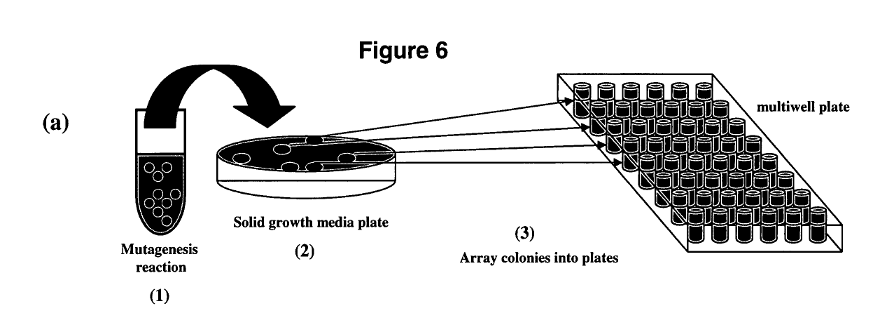

Genomic diversity within microbes can be induced using techniques such as chemical mutagenesis (such as nitrosoguanidine, UV light exposure, EMS, and other mutagens (see for example Zhang et al., Nature 2002 Feb. 7;415(6872):644-6; Cell Mol Biol Lett. 2003;8(2):261-8.). A single, homogeneous strain of a microorganisms can be used to make a library of genomically diverse microorganisms by mutagenizing a population of cells, plating the survivors on solid media, and picking independent colonies. See FIG. 6.

Genomic diversity can also be induced by transforming strains with nucleic acid constructs such as promoters. Because each construct lands in a different part of the genome, each cell that acquires an integrated construct can exhibit a different phenotype. Any difference in genome sequence between two cells is genomic diversity. Methods of transforming microbes are well known in the art (see, e.g. (Harris, (1989) The Chlamydomonas Sourcebook. Academic Press, New York); Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)).

In one embodiment, promoter sequences from a plurality of genes in the genome of an organism are used to transform cells, followed by screening or selection for a desired phenotype. For example, a plurality of 500 base pair, 1000, 1500, 2000, or more base pair promoters from different genes are amplified from the C. reinhardtii genome. The full genome sequence has been completed and are found at http://genome.igi-psf.org/chlre1/chlre1.home.html. The amplified promoter sequences are attached to a selectable marker sequence and used to transform the nuclear and/or chloroplast and/or mitochondrial genome of Chlamydomonas reinhardtii, other Chlamydomonas species, or other green algae. Each independent transformant is genomically diverse from the other independent transformants.

A second microorganism is derived from a first microorganism through mutagenesis and/or nucleic acid exchange of the first microorganism (and another genomically diverse third microorganism if the method is nucleic acid exchange).

Microarrays

Microarrays for microbial genomes such as Chlamydomonas reinhardtii, E. coli, S. cerevisae, and many others are available. These microarrays can contain genomic sequence, or preferably, cDNA sequences. For examples, see Plant Physiol. 2003 February;131(2):401-8, Nat Biotechnol. 2004 January;22(1):86-92; Antimicrob Agents Chemother. 2004 March;48(3):890-6; J Biol. Chem. 2003 Sep. 12;278(37):34998-5015.

Microarrays are synthesized using well known methods. (See, eg, U.S. Pat. Nos. 6,566,495; 5,919,523; 6,239,273).

Microarrays are designed as described in example 1. The codon usage regime of an organism can be determined by sequencing a relatively small number (˜20) of cDNAs. Many codon usage regimes are known. For examples, see http://www.kazusa.orjp/codon/.

The following example is provided by way of illustration and is not intended as limiting. Any trait can be optimized using the methods disclosed herein. The following example contains methods for optimizing hydrogen production in green algae.

### EXAMPLE 1

1. Genomic Proxy Microarrays

Microarrays containing all known cDNA sequences of C. reinhardtii can be commercially obtained (see Carnegie Institute/Duke/Stanford Genome Technology Center—Chlamydomonas MicroArray Project at http://aracyc.stanford.edu/˜jshrager/lab/chlamyarray/). Although the genome sequence of only C. reinhardtii has been elucidated (and not other Chlamydomonas species), this genomic information is used to quantitate expression levels of genes in any microbial species, preferably with the genus Chlamydomonas. This is accomplished by creating genomic proxy microarrays for other Chlamydomonas species.

Genomic proxy microarrays are generated by translating the approximately 9,000 C. reinhardtii cDNA sequences into protein sequences, followed by reverse translating the sequences into cDNAs that utilize the codon preferences of a different Chlamydomonas species. Table 4 contains accession numbers for C. reinhardtii cDNAs that can be reverse translated. The correct reading frame is deduced by both (a) comparing the nucleotide sequence to databases using programs such as BLAST (http://www.ncbi.nlm.nih.gov/BLAST), and (b) by translating the sequence using all 6 reading frames (each frame in both directions) and comparing the translated protein sequence against sequence databases such as Swiss-PROT.

For example, the C. reinhardtii cDNA sequences are converted to C. pallidostigmatica codon usage without altering the sequence of any proteins encoded by the cDNAs. This process is used to generate microarray sequence sets that are optimized to any particular Chlamydomonas species for which the codon usage regime is known. The complete set of codon-optimized cDNAs are spotted onto microarrays, where a distinct microarray is designed for each species that has a distinct codon usage regime. Each specific spot on all microarrays corresponds to the same protein sequence regardless of the divergent nucleotide sequences immobilized on the respective spots. In other words, a series of microarrays are created that contain immobilized cDNA sequences written in the codon usage preference of distinct species of Chlamydomonas. It is worthwhile noting that this process need not be performed for non-reinhardtii species that have the same codon usage preferences as C. reinhardtii. For example, the codon usage preferences of C. moewusii and C. incerta are essentially identical to C. reinhardtii (see http://www.kazusa.or.jp/codon).

Microarrays are synthesized that correspond to all known expressed C. reinhardtii proteins. A first microarray contains immobilized, single-stranded DNA molecules corresponding to all known C. reinhardtii cDNA sequences. In a preferred embodiment, the immobilized cDNA sequences are chemically synthesized in segments corresponding to overlapping sections of each cDNA, each segment being immobilized on positions next to each other on the array. This is preferred since sequences longer than about 100 nucleotides are difficult to chemically synthesize accurately. During data analysis the overall expression level of a gene is calculated by adding the labeling intensity of all spots corresponding to a single cDNA.

A second microarray contains single-stranded DNA molecules encoding the exact same set of protein sequences as the first microarray. However, the second microarray encodes the protein sequences using C. pallidostigmatica degenerate, most preferred codons. For example, the amino acid alanine is encoded by C. pallidostigmatica using almost exclusively by the codons GCT and GCA. An alanine codon on the microarray is synthesized as G-C-T/A, where the third position are synthesized using a mixture of thymine and adenine. Additional microarrays are constructed using the codon usage regimes of the other H₂-producing Chlamydomonas strains listed in tables 2 and 3. Because the codon usage regimes of species such as C. moewusii are essentially identical to C. reinhardtii, standard C. reinhardtii microarrays are used to analyze these species.

Microarray expression analysis is performed by isolating mRNA from cells that are performing a particular metabolic function, in this case photoproducing H₂. Labeled nucleic acids are then derived from the mRNA. For example, the mRNA is reverse transcribed into cDNA, which is fluorescently labeled by the incorporation of labeled deoxynucleotides in the reverse transcription reaction. The labeled cDNA is then fragmented to a size of approximately 30-40 nucleotides (see e.g., Affymetrix GenChip® Expression Analysis Handbook version 701021 rev 1). Fragments that correspond to highly conserved domains of proteins specifically anneal to their complementary, immobilized sequences. This specific annealing quantitates the level of transcription of a gene regardless of the lack of specific annealing of other fragments to regions of the same immobilized gene. The labeled cDNA is an example of a nucleic acid derivative of the mRNA sample. Other nucleic acid derivatives are PCR-generated fragments and propagated vectors.

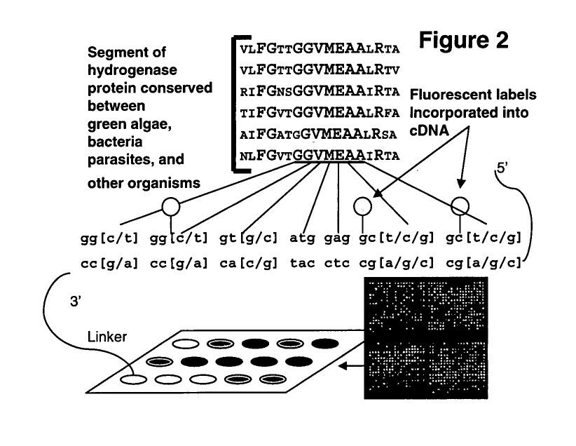

As an example, a section of the iron hydrogenase protein is depicted in FIG. 2. This enzyme catalyzes the formation of H₂ produced by Chlamydomonas. The sequence motif GGVMEAA is highly conserved in iron hydrogenase proteins across numerous species. The amino acid comparison depicted in FIG. 2 shows, from top to bottom, this region of the iron hydrogenase proteins from Chlamydomonas reinhardtii (green algae), Scenedesmus obliquus (green algae), Megasphaera elsedenii (bacteria), Desulfovibrio desulfuricans (bacteria), Clostridium pasteurianum (bacteria), and Nyctotherus ovalis (ciliate). An immobilized hydrogenase cDNA fragment encoded by the degenerate, preferred codon usage regime of Chlamydomonas pallidostigmatica provides an annealing target for labeled fragments of C. pallidostigmatica cDNA. The annealing of fragments to cDNA regions that correspond to highly conserved amino acid motifs occurs in a quantitative fashion relative to the level of C. pallidostigmatica hydrogenase gene expression, regardless of the level of annealing of labeled fragments to less conserved regions. The result is that intraspecies expression comparisons between different strains of genomically diverse microorganisms are highly quantitative. In other words, the extent of annealing of each labeled cDNA fragment to a non-reinhardtii microarray is constant between all the strains of a single species. Any bias to increase or decrease the likelihood of annealing of a particular fragment of labeled cDNA from a particular strain is identical for all strains of the same species. Synthesizing degenerate immobilized cDNA sequences that reflect preferences for more than one codon for a particular amino acid increases the likelihood of a particular fragment annealing to its immobilized complement on a microarray. For example, all immobilized C. pallidostigmatica microarray cDNA sequences contain a mixture of GCT and GCA codons for each position that encodes an alanine (see FIG. 1(b)). Synthesis of such immobilized, degenerate probes at a single spot on a microarray is a well-established technique.

The intraspecies expression data obtained from numerous distinct strains of numerous distinct species of Chlamydomonas from tables 2 and 3 is then correlated with measurements of the amount of H₂ produced by each strain of each species before mRNA samples are taken, preferably shortly (such as 20 minutes or less) before mRNA samples are taken. If the expression level of a gene directly affects H₂ production, this fact is detected by the microarray data analysis. For example, when the average expression level of a gene is significantly higher in more productive strains than in less productive strains of the same species the gene is an opportune target for H₂ production enhancement. Opportune targets for H₂ production enhancement are tested by linking them to heterologous promoters, as described below. Note that other hydrogen producing green algae cited in Brand et al. Table II are also preferred organisms for analysis using genomic proxy microarrays based on the C. reinhardtii genome.

2. H₂ Production (Desired Function) Assay and Isolation of mRNA Samples from Genomically Diverse Microorganisms

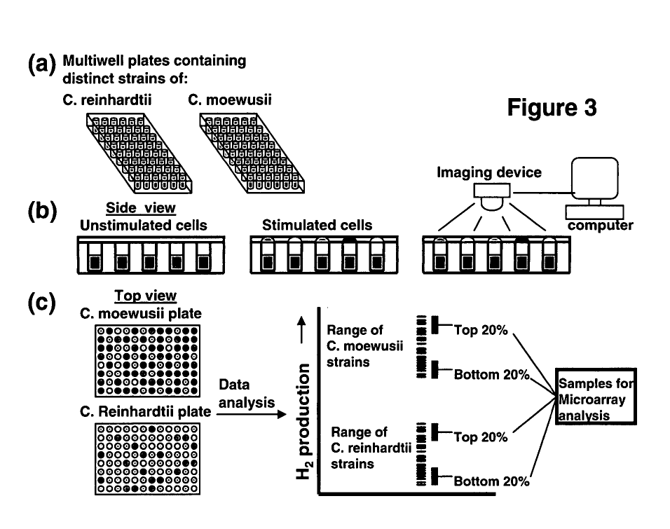

As depicted in FIGS. 3(a)-(c), the hydrogen production assay is performed by arraying distinct strains of different Chlamydomonas species in multiwell plates. The chemochromic film is placed over the plates. The cells are then stimulated to photoproduce H₂ using methods such as depriving the cells of sulfur (Plant Physiol. 2000 January;122(1):127-36). As H₂ gas is generated, it bubbles to the surface of the culture media, fills the gas space, and forms reversible complexes with metal atoms in the film. The coordination of H₂ molecules and metal atoms creates a dark spot on the film in a quantitative fashion relative to the amount of H₂ present (see U.S. Pat. Nos. 6,448,068 and 6,277,589). An image of the film is captured and downloaded to a computer that quantitates the relative intensity of each spot on the film, identifying different levels of H₂ production between wells. The data is then analyzed to identify the range of production exhibited between different strains of the same species, as shown in FIG. 3(c). Cells can also be stimulated to produce H₂ (or perform another desired function) using a plurality of distinct induction methods.

3. Microarray Data Acquisition/Analysis to Identify Opportune Targets

Shortly after H₂ measurement, mRNA is isolated from strains, preferably in a parallel isolation procedure. The mRNA are reverse transcribed, labeled, and fragmented. Samples are applied to codon-optimized genomic proxy microarrays corresponding to each species. A full set of expression data is obtained for each strain. Expression differences are analyzed for each gene on the microarray for each strain to generate a range of expression of the gene from the high and low H₂ producers.

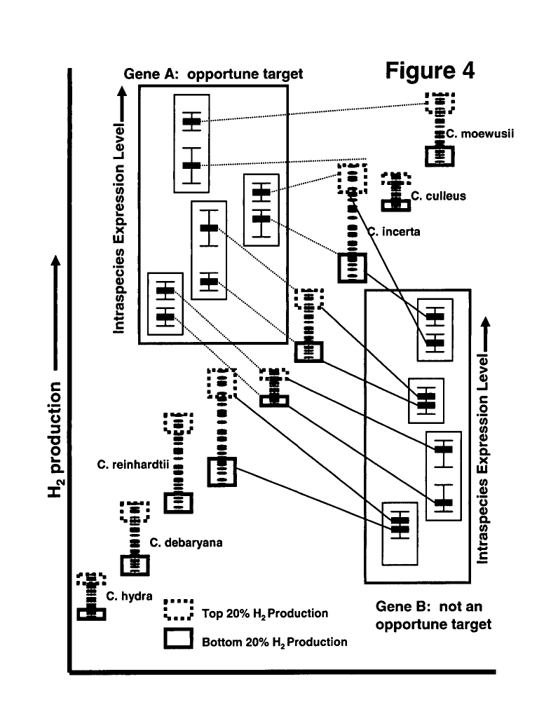

The data analysis is depicted in FIG. 4. All strains of each species are stratified according to H₂ production levels. In one embodiment, the top 20% of H₂ producers of each species and the bottom 20% of H₂ producers of each species are subjected to expression analysis using the appropriate microarray for each species. Selection of more than simply the absolute highest and lowest producing strain of each species produces more data points that are used to distinguish significant differences in expression levels from artifacts. The percent of strains analyzed as high and low producers can be any percent of the total strains analyzed, not just 20%. For example the top and bottom 1%, 5%, 10%, and 35% can be assayed. It is not necessary to select the same percent higher and lower strain numbers.

Two genes are depicted for analysis in FIG. 4. For clarity, the expression levels are only depicted in FIG. 4 for a subset of species, however all species tested are preferably subjected to expression analysis. In each species, the expression level of gene A is higher in the top H₂ producers than in the bottom H₂ producers for each species. Gene A is therefore an opportune target for expression in transformed test strains to generate novel strains of Chlamydomonas that produce higher levels of H₂ than a contol, non-tranformed test strain. The expression levels of gene B, however, have no relationship to H₂ production. Gene B is not an opportune target.

Table 3 contains genetically distinct species of C. reinhardtii that were isolated from geographically diverse locations. The top and bottom 20% of H₂ producers from this collection corresponds to a total of 16 strains. To increase statistical power, a mixture of cells from all 40 C. reinhardtii strains can be subjected to random (SNP-inducing) chemical mutagenesis to create a library of 200 genetically diverse strains that have a broader intraspecies range of H₂ production. The top and bottom 20% of H₂ producers from the new library corresponds to 80 strains. The increase in the number of strains with a corresponding increase in genetic diversity and range of H₂ production levels allows more statistically significant data to be obtained. This leads to the identification of more legitimate opportune targets because the likelihood of an expression pattern such as that shown for gene A of FIG. 4 being generated by chance is reduced as the number of strains analyzed increases. Therefore, preferably at least 20, more preferably 40, more preferably 200, and more preferably 500 or more distinct strains of each species are analyzed.

These new libraries described above can also be used in other methods to optimize any desired function of Chlamydomonas, not just H₂ production.

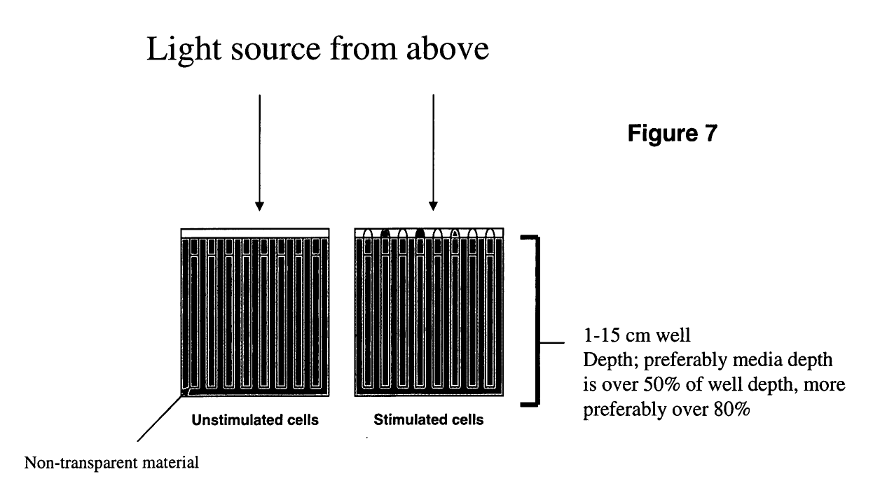

One embodiment of screening a library of cells is using the assay system of FIG. 7. The cells are placed in deep well plates of at least 1, 2, 3, 4, 5, 6, 7, 8, 9, 10, 11, 12, 13, 14, 15, or more cm in depth. The deep well plates are made of a non-transparent material that does not transmit light or transmits a significantly reduced amount of light compared to transparent material. The light source provides light only from directly above. A library is constructed using a mutagenesis technique and cells are screened for the ability to make increased levels of H₂ compared to the starting strain used to make the library. Preferably the light source is bright, meaning that it delivers enough photons to cells that wild type C. reinhardtii cells in the deep wells dissipate at least 50%, more preferably 80%, more preferably 90% or more of the light energy absorbed by light harvesting antennae as heat. Strains in deep wells that produce more H₂ than wild type have increased photon utilization efficiency and are advantageous for commercial hydrogen production because they do not waste absorbed light at the same level as wild type C. reinhardtii. Preferably strains identified using this technique waste less than 10% of absorbed light under bright conditions. These preferred cells do not block photons from penetrating deeper into the media and being harvested by cells not directly at the surface of the media.

4: Construction and Expression of Opportune Target Test Expression Vectors

Opportune targets expressed at higher levels in cells of at least 1 species, preferably 2 species, more preferably 3 species, more preferably 4 species, and so on, that produce higher amounts of H₂ and are expressed at lower levels in strains of these species that produce lower amounts of H₂ are synthesized as cDNA sequences for test expression. These genes are referred to as opportune targets. Opportune targets are expressed in host strains and increases in H₂ production are assayed using the screening system depicted in FIG. 3.

In green algae, the nuclear, mitochondrial, and chloroplast genomes are transformed through a variety of known methods. (Kindle, J Cell Biol (1989) Dec;109(6 Pt 1):2589-601; Kindle, Proc Natl Acad Sci USA (1990) Feb;87(3):1228-32; Kindle, Proc Natl Acad Sci U S A (1991) Mar 1;88(5):1721-5; Shimogawara, Genetics (1998) Apr;148(4):1821-8; Boynton, Science (1988) Jun 10;240(4858):1534-8; Boynton, Methods Enzymol (1996) 264:279-96; Randolph-Anderson, Mol Gen Genet (1993) Jan;236(2-3):235-44).

Selectable markers for use in Chlamydomonas are known, including but not limited to markers imparting spectinomycin resistance (Fargo, Mol Cell Biol (1999) Oct;19(10):6980-90), kanamycin and amikacin resistance (Bateman, Mol Gen Genet (2000) Apr;263(3):404-10), zeomycin and phleomycin resistance (Stevens, Mol Gen Genet (1996) Apr 24;251(1):23-30), and paromycin and neomycin resistance (Sizova, Gene (2001) Oct 17;277(1-2):221-9).

Screenable markers are available in Chlamydomonas, such as the green fluorescent protein (Fuhrmann, Plant J (1999) Aug;19(3):353-61) and the Renilla luciferase gene (Minko, Mol Gen Genet (1999) Oct;262(3):421-5). Fluorescent proteins are also available for prokaryotic organisms.

Cell transformation methods and selectable markers for photosynthetic bacteria and cyanobacteria are well known in the art (Wirth, Mol Gen Genet 1989 March;216(1):175-7; Koksharova, Appl Microbiol Biotechnol 2002 February;58(2):123-37; Thelwell). Transformation methods and selectable markers for use in bacteria are well known (Maniatis et al. (1989) Molecular Cloning: A Laboratory Manual Cold Spring Harbor Laboratory).

The opportune targets can be expressed by heterologous promoters. Heterologous promoters are promoters other than the natural, endogenous promoter that activates the opportune target in its genomic location in a wild-type organism. Heterologous promoters can come from the same organism (C. reinhardtii) and still be considered heterologous when they activate an opportune target other than the gene they activate in wild-type cells.

Preferably, the opportune targets are driven by light-activated promoters. Numerous light-activated C. reinhardtii promoters are known. Light-activated C. moewusii genes are isolated by differential expression analysis using C. reinhardtii microarrays and C. reinhardtii cells (a) in the dark and (b) exposed to 20 minutes of light. Light-induced promoters are isolated and a validated promoter is selected. Opportune targets can be driven by any type of promoter, such as inducible or constitutive promoters. For example, in Chlamydomonas, a promoter sequence that imparts transcriptional activation when a cell is exposed to light may be incorporated into the vector (for examples see Hahn et al., Curr Genet (1999) Jan;34(6):459-66, Loppes et al., Plant Mol Biol 2001 January;45(2):215-27, and Villand et al. Biochem J 1997 Oct. 1;327 (Pt 1):51-7). Other light-inducible promoter systems may also be used, such as the phytochrome/PIF3 system (see Shimizu-Sato et al., Nat Biotechnol 2002 October;20(10):1041-4). Other promoters may be used that activate expression when a cell is exposed to light and heat (for examples, see Muller et al., Gene (1992) Feb 15;111(2):165-73, von Gromoff et al., Mol Cell Biol (1989) Sep;9(9):3911-8). Other promoters may be used that activate expression when a cell is exposed to darkness (for example, see Salvador et al., Proc Natl Acad Sci USA 1993 Feb. 15;90(4): 1556-60). Alternatively the promoter sequence imparts transcriptional activation when an exogenous molecule is added to the culture media using receptors not present in the wild-type cell such as receptors for estrogen, ecdysone, or others (Metzger et al., Nature 1988 Jul. 7;334(6177):31-6, No et al. Proc Natl Acad Sci USA 1996 Apr. 16;93(8):3346-51). Alternatively a constitutive promoter can be used such as the promoter of the RBCS2 or psaD genes (see Stevens et al., Mol Gen Genet (1996) Apr 24;251(1):23-30 and Fischer, WO 01/48185).

It should be noted that genes that siphon resources away from the H₂ production pathway may also be downregulated in an inverse manner to the opportune targets discussed above. In other words, their expression patterns may be inverse to those depicted for gene A in FIG. 4. siRNA constructs are designed for genes that exhibit this inverse pattern of expression. The siRNA genes are preferably expressed by light-activated promoters discussed above. siRNA technology is known (For examples, see Fire et al., Nature (1998) Feb 19;391(6669):806-11 and Fuhrmann et al., J Cell Sci (2001) Nov;114(Pt 21):3857-63).

Transformed test strains containing opportune target expression vectors that produce more H₂ than the control, non-transformed test strains are selected for further development described below, and are also referred to as validated transformed test strains. In one embodiment a C. reinhardtii strain is the host test strain used to construct transformed test strains. The opportune target can be a cDNA sequence from any organism; in other words, because multiple species and strains within a species are used in the methods described herein, any cDNA having significant sequence identity with a cDNA identified in the microarray analysis can be used as an opportune target to be expressed. Preferably an opportune target contains sequence that uses the same or similar codon usage regime of a host test strain. Opportune target coding regions can be expressed in a naturally occurring cDNA sequence or as a synthetic gene.

5: Nucleic Acid Exchange to Concentrate Validated Opportune Target Expression Vectors

The metabolic pathway of H₂ production in C. reinhardtii is complex, and functions through dynamic interactions between genes and gene products distributed throughout all three genomes of the organism (nuclear, chloroplast, and mitochondrial). The benefit of all possible expressed opportune targets is reaped by mating a transformed test strain containing opportune target expression vector that produces more H₂ than the control, non-transformed test strain with at least one other transformed test strain containing a different opportune target expression vector that produces more H₂ than the control, non-transformed test strain, and screening for progeny from the mating that produce more H₂ that any parental strain.

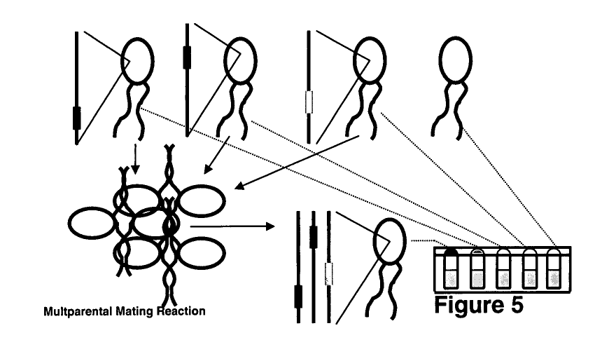

Preferably, all validated transformed test strains are placed together in mating reactions. Mating protocols for organisms such as green algae are also known (Harris, (1989) The Chlamydomonas Sourcebook. Academic Press, New York; and in U.S. patent application Ser. No. 10/763,712). Although a C. reinhardtii cell is only capable of mating with one other cell at a time, multiple improved strains are placed into the same mating reaction. In a multiparental mating reaction, 3 or more distinct strains are put through multiple cycles of mating. In each cycle, the cells mate with the progeny of other mating events from earlier cycles. This process results in a small percentage of progeny strains in the mating reaction accumulating a large number of expression vectors containing opportune targets through cosegregation and recombination events, as depicted in FIG. 5. The multiparental mating reaction can then be plated out as a library. Each individual progeny colony is arrayed into multiwell plates and all progeny from the mating reaction are assayed. Progeny strains that produce more H₂ than any parental strain are then selected for further mating with other improved strains.

The mating process can also be performed in a pairwise fashion, where only two improved strains at a time are systematically mated with each other and the progeny are screened.

Preferably, all validated opportune target expression vector-containing strains put through the above described assay and mating process in an iterative fashion until 5, 10, 20, or more, and/or all validated opportune target expression vectors are contained in a single strain to be deployed for commercial hydrogen production.

It should be apparent to one skilled in the art that various embodiments and modifications may be made to the invention disclosed in this application without departing from the scope and spirit of the invention. All publications mentioned herein are cited for the purpose of describing and disclosing reagents, methodologies and concepts that may be used in connection with the present invention. Nothing herein is to be construed as an admission that these references are prior art in relation to the inventions described herein. All references cited are hereby incorporated by reference for all purposes.

TABLE 4

| | | | |
|---|---|---|---|
| BE024923 | BF862366 | BE724264 | BE238098 |
| BF866577 | BE452474 | BF865931 | BE129080 |
| BE453278 | BE726006 | BE725927 | BE121859 |
| BE351940 | BE726630 | BE453128 | BF866824 |
| BE452548 | BE452152 | BF859706 | BE725222 |
| BE761243 | BE452207 | BF860823 | BE725377 |
| BE238392 | BF866418 | BE337707 | BE212255 |
| BE227513 | BF861938 | BE024764 | BE024853 |
| BE761252 | BF863773 | BE761358 | BE025164 |
| BF866008 | BF862769 | BE726826 | BE452751 |
| BE725340 | BE724441 | BE725706 | BE453546 |
| BE351718 | BE122244 | BE352144 | BF864032 |
| BE122041 | BF862306 | BE056528 | BE056664 |
| BF866095 | BF866782 | BF859320 | BE725998 |
| BE724448 | BE724654 | BE121448 | BE725841 |
| BE725039 | BF863270 | BE726379 | BE724414 |
| BE211823 | BE453166 | BE453405 | BE724532 |
| BE024352 | BE452278 | BE452615 | BE212024 |
| BF861883 | BF863960 | BF866559 | BE212109 |
| BF861543 | BE337246 | BF867372 | BF862625 |
| BE121846 | BE129374 | BE227663 | BE452692 |
| BE351830 | BF863958 | BE238413 | BE453203 |
| BE453417 | BE129115 | BF863186 | BE452648 |
| BE452511 | BF863759 | BE227377 | BE452146 |
| BF862075 | BF862313 | BF867216 | BE337127 |
| BE724349 | BF863663 | BF862065 | BF860907 |
| BE025029 | BE724969 | BF866563 | BE337135 |
| BE453415 | BE056738 | BE211787 | BE121543 |
| BF864480 | BE726676 | BE024155 | BE121933 |
| BF861725 | BE238390 | BF862561 | BE724967 |
| BF863719 | BE724365 | BE352100 | BF862812 |
| BF860235 | BE337201 | BF866517 | BE761412 |
| BF861999 | BE238109 | BE056534 | BF867592 |
| BE351910 | BE237914 | BE024515 | BE724656 |
| BE129013 | BF861754 | BE725405 | BE453338 |
| BE238346 | BF861812 | BE238232 | BE725816 |
| BE452667 | BE237739 | BE725843 | BE237902 |
| BE761213 | BE056678 | BE238416 | BF860556 |
| BE351716 | BE452937 | BF863038 | BE025112 |
| BF859858 | BE352052 | BF860402 | BE452723 |
| BE725829 | BF866194 | BF866499 | BE238032 |
| BE056462 | BE122083 | BE337133 | BE337139 |
| BE024217 | BE452353 | BF862101 | BE024782 |
| BE227931 | BE453205 | BE725905 | BE726129 |
| BF862677 | BF864458 | BE725026 | BF862862 |
| BE352094 | BE453366 | BE129430 | BE724294 |
| BF863200 | BE122390 | BE129402 | BF859887 |
| BE725283 | BE453556 | BE129407 | BF863090 |
| BE024595 | BF862481 | BE024906 | BE352294 |
| BE337215 | BE024483 | BF862432 | BE724426 |
| BF866742 | BE453467 | BF866293 | BE724585 |
| BF863234 | BE726520 | BE452849 | BE725129 |
| BF860939 | BE761468 | BE129056 | BE725664 |
| BF866730 | BF863332 | BF863541 | BE056740 |
| BE121835 | BE238400 | BF863855 | BE122014 |
| BE056585 | BE724304 | BF860045 | BE024660 |
| BE024900 | BE453424 | BF859301 | BE352085 |
| BE227701 | BE761216 | BE337125 | BE453554 |
| BE337640 | BE724306 | BF860791 | BF863908 |
| BE238429 | BF862169 | BF860580 | BE352090 |
| BE337686 | BE453438 | BE024952 | BF863645 |
| BE227691 | BE238127 | BF862904 | BF866989 |
| BF863853 | BF863134 | BE024856 | BF860856 |
| BE129492 | BE024651 | BE452455 | BF859179 |
| BF866641 | BE337191 | BF860786 | BE724522 |
| BE452945 | BF861741 | BF863428 | BF863144 |
| BE238153 | BE121841 | BE056798 | BF863535 |
| BE452375 | BF865925 | BF863865 | BE129344 |
| BE452735 | BF863102 | BF863346 | BE337273 |
| BE025146 | BE129000 | BF863898 | BE227643 |
| BE024808 | BE211923 | BE024825 | BE238198 |
| BE724348 | BE351832 | BE129250 | BE238006 |
| BE122285 | BF863629 | BE452516 | BE129242 |
| BF861567 | BF863667 | BE024961 | BE121509 |
| BE238419 | BE725613 | BF863972 | BF864000 |
| BF862376 | BE725041 | BE129029 | BE129268 |
| BE724431 | BE725461 | BE726811 | BF863603 |
| BE212221 | BF859750 | BE212376 | BF862713 |
| BE725229 | BE725837 | BE453196 | BE452157 |
| BE238204 | BE024381 | BF861519 | BE024452 |
| BE453056 | BE726398 | BE024498 | BF859675 |
| BE056772 | BF866016 | BF860678 | BF864038 |
| BE453177 | BE122078 | BE212074 | BE212317 |
| BE453226 | BE238212 | BE452229 | BE025009 |
| BE211934 | BE024628 | BE725688 | BE725258 |
| BE453552 | BE024937 | BE725350 | BE452595 |
| BE238314 | BE453099 | BE452950 | BE725756 |
| BE725501 | BF863851 | BE237697 | BE726116 |
| BE212152 | BE351852 | BE726322 | BE024569 |
| BE725268 | BF862465 | BE725206 | BF861758 |
| BF864865 | BE453360 | BE726309 | BE725063 |
| BF862600 | BE351692 | BE452509 | BF864284 |
| BE211866 | BE724263 | BE227520 | BF862982 |
| BF865948 | BE725735 | BE238248 | BE351834 |
| BE726928 | BE129246 | BE129171 | BE351793 |
| BE352255 | BF863721 | BE352136 | BE452644 |
| BE212372 | BF863218 | BE024408 | BF863434 |
| BE351881 | BE227426 | BE129476 | BE725330 |
| BF860601 | BE024817 | BE452853 | BF867608 |
| BE452187 | BF866671 | BE212315 | BE724313 |
| BE056715 | BF863216 | BE237720 | BE726685 |
| BE726749 | BE352018 | BF859834 | BE227412 |
| BE724817 | BF866724 | BE227482 | BE227391 |
| BE024860 | BE129452 | BF859671 | BF860175 |
| BF862499 | BE351909 | BE351888 | BF863318 |
| BE725373 | BF862211 | BE761448 | BE056536 |
| BE237786 | BE726480 | BF860418 | BE211939 |
| BE337230 | BF863196 | BE056552 | BF867647 |
| BE129409 | BE726536 | BE725655 | BF862179 |
| BE452922 | BE726375 | BF860802 | BE453462 |
| BF860323 | BE453218 | BE227772 | BE452744 |
| BE237703 | BE725823 | BE237934 | BE725085 |
| BF860089 | BF859891 | BE337573 | BE129058 |
| BE724985 | BE725047 | BE122355 | BE351937 |
| BF860292 | BF861691 | BE227444 | BE761229 |
| BE337128 | BE238218 | BF863799 | BF862491 |
| BE237846 | BE056621 | BE129271 | BE238383 |
| BF861948 | BE452416 | BE211776 | BE725996 |
| BF859796 | BF860510 | BE351901 | BE237727 |
| BE452258 | BE726434 | BE129518 | BE725362 |
| BE227682 | BE452822 | BE726706 | BE724564 |
| BE725977 | BF863472 | BE227476 | BE726885 |
| BE227749 | BF862777 | BE122206 | BE337170 |
| BF863096 | BE453311 | BF866969 | BE351763 |
| BF863444 | BF864263 | BE227716 | BE724895 |
| BF863326 | BE725348 | BE351988 | BE724949 |
| BF862209 | BF866981 | BE725987 | BE211901 |
| BE453444 | BE724644 | BE726438 | BE237663 |
| BE724695 | BE725173 | BE024336 | BE129372 |
| BE725979 | BF860488 | BE129083 | BF860468 |
| BE238328 | BE724528 | BE725892 | BE122246 |
| BE122296 | BF863807 | BE453422 | BF862647 |
| BE212213 | BE724871 | BE725645 | BF863452 |
| BE121985 | BF864686 | BF859770 | BE024511 |
| BE453305 | BF862988 | BF863902 | BE453328 |
| BF866551 | BF862195 | BE352010 | BE056380 |
| BE024862 | BE453536 | BF864369 | BE024272 |
| BF863679 | BE724526 | BF864612 | BE725028 |
| BE452898 | BE725737 | BF866720 | BE352289 |
| BF863408 | BE726114 | BE238288 | BF862392 |
| BF862529 | BF863264 | BE453575 | BF862083 |
| BF863813 | BF861770 | BE452779 | BE761517 |
| BE726361 | BF866651 | BE453143 | BE351820 |
| BE121593 | BE453126 | BE725885 | BF861709 |
| BE351646 | BE227527 | BE351972 | BE724424 |
| BE024172 | BF860564 | BE452717 | BE122322 |
| BE227676 | BE024975 | BF866379 | BE724951 |
| BE025013 | BE724902 | BE238269 | BE238055 |
| BE725473 | BE725135 | BE724359 | BF865992 |
| BE452900 | BE237996 | BE452543 | BE453183 |
| BE452534 | BE726248 | BE351886 | BE212145 |
| BE352161 | BE351706 | BF861505 | BE129125 |
| BE452167 | BE453629 | BE452731 | BF862424 |
| BE024433 | BF863458 | BE724280 | BE238183 |
| BF860760 | BE351778 | BE725143 | BF863884 |
| BE122271 | BF863064 | BF863424 | BF863244 |
| BE352229 | BE227592 | BE452719 | BE337603 |
| BF866891 | BE024870 | BE726530 | BE025000 |
| BF864048 | BE129252 | BE725371 | BE227457 |
| BE452141 | BF861794 | BE725394 | BF859762 |
| BF860496 | BE237866 | BE024783 | BE211880 |
| BF860496 | BE724652 | BE452351 | BE056559 |
| BE024979 | BE212381 | BF863880 | BF863910 |
| BE725689 | BE725709 | BF860451 | BE122252 |
| BE337599 | BE025105 | BE122029 | BE351998 |
| BE452827 | BE212133 | BE237743 | BF862019 |
| BF862511 | BE227396 | BE128970 | BF861920 |
| BE227785 | BE453340 | BE726522 | BF860775 |
| BF863952 | BF866133 | BE337209 | BE237823 |
| BF863559 | BE121989 | BE129460 | BE129293 |
| BE129277 | BE725388 | BE761481 | BF861802 |
| BE453152 | BE452163 | BE121612 | BE237984 |
| BE725250 | BE024521 | BE726046 | BE351847 |
| BE453488 | BF860436 | BF863228 | BE237800 |
| BF862795 | BF863166 | BE025116 | BE129127 |
| BE212080 | BE211895 | BE724266 | BE212274 |
| BE337203 | BE122085 | BE452311 | BF864446 |
| BF863918 | BE024985 | BE725683 | BE024575 |
| BE726082 | BE129395 | BF861863 | BE025200 |
| BE212304 | BE337579 | BF864528 | BF862824 |
| BE121991 | BE056397 | BF862147 | BE453564 |
| BE121647 | BE352338 | BE227473 | BF864016 |
| BE129528 | BE725806 | BE024915 | BE238041 |
| BF860655 | BE238368 | BF862276 | BE237745 |
| BE725106 | BE025144 | BF862602 | BF861669 |
| BE238344 | BE351965 | BE227603 | BF867354 |
| BE452334 | BE056595 | BF862493 | BE761259 |
| BE351953 | BE724983 | BE238023 | BE212364 |
| BE024666 | BF862693 | BF862844 | BE452359 |
| BE056654 | BE024769 | BF861353 | BF859715 |
| BE237848 | BE724533 | BE352337 | BE453542 |
| BF861637 | BE129393 | BF862241 | BE761521 |
| BE452764 | BE725303 | BE452796 | BE452574 |
| BE212175 | BF864544 | BE129279 | BE761351 |
| BF860275 | BF863605 | BF862856 | BE724959 |
| BF862709 | BE725391 | BF862445 | BE724267 |
| BE227465 | BE724681 | BE725311 | BF865990 |
| BE238278 | BF866369 | BE337678 | BF862884 |
| BE724418 | BE724299 | BE211870 | BE212387 |
| BF866004 | BE453469 | BE122016 | BE725104 |
| BF863396 | BE726939 | BE725253 | BE129502 |
| BE452233 | BF866987 | BE122147 | BF862727 |
| BF863609 | BE227604 | BE122181 | BE761320 |
| BE452701 | BE129299 | BE227471 | BE724356 |
| BE121746 | BE024845 | BE352140 | BE227463 |
| BE025180 | BE056384 | BE761433 | BE129546 |
| BE351949 | BE237671 | BE212169 | BE024814 |
| BF863585 | BE122372 | BE212219 | BF866165 |
| BF863819 | BE453108 | BE725677 | BE212289 |
| BE725245 | BE725860 | BE024542 | BE725907 |
| BF862651 | BE238043 | BF862972 | BF861418 |
| BE726604 | BE212261 | BE452324 | BF863673 |
| BE024806 | BE122408 | BE056819 | BE237870 |
| BE337259 | BE351969 | BE121681 | BF863529 |
| BE024625 | BE129129 | BE761293 | BE227511 |
| BE453606 | BE351688 | BE121927 | BE237804 |
| BF862457 | BF860560 | BE761196 | BF862459 |
| BE129212 | BE212057 | BE724942 | BF863749 |
| BE129121 | BF860592 | BE725139 | BF862569 |
| BE024930 | BE237699 | BE238325 | BE121880 |
| BE121995 | BE024404 | BE056680 | BE024439 |
| BE024872 | BE725252 | BF865887 | BE725784 |
| BF863876 | BE237762 | BE726790 | BF861788 |
| BE212238 | BE238381 | BE726598 | BE453286 |
| BF859909 | BF861885 | BE211904 | BF863797 |
| BE725479 | BE352200 | BE725649 | BE725269 |
| BF864012 | BF863849 | BE726381 | BE724885 |
| BE212196 | BF860021 | BE726637 | BE761527 |
| BE238019 | BE724272 | BE726660 | BE726506 |
| BF862463 | BF862797 | BE211827 | BE724569 |
| BE337113 | BE352130 | BE726528 | BE121749 |
| BE724310 | BE761290 | BF862037 | BF865933 |
| BE726202 | BF863886 | BE227805 | BE211893 |
| BF861447 | BE211825 | BF863314 | BE056743 |
| BF862814 | BE121711 | BE227402 | BE725344 |
| BE122019 | BE725277 | BE724878 | BE726041 |
| BE725010 | BE761211 | BE726876 | BF864010 |
| BE725337 | BE211991 | BE726048 | BE056445 |
| BF863841 | BE453181 | BE238192 | BE352054 |
| BE724837 | BE726314 | BF860036 | BE024602 |
| BE725831 | BE724626 | BF865972 | BE725690 |
| BE237821 | BF861365 | BE238373 | BF863577 |
| BE024995 | BE453457 | BE351710 | BE352103 |
| BE129100 | BF861559 | BF861408 | BE237929 |
| BE122110 | BE211938 | BF859901 | BE238297 |
| BF862872 | BE238335 | BE238277 | BE352202 |
| BE337252 | BE724377 | BF863615 | BE121672 |
| BE452682 | BE453245 | BE453074 | BE453282 |
| BF862031 | BF860544 | BE452910 | BF859983 |
| BE211993 | BE724658 | BE725147 | BE227393 |
| BE724460 | BE761206 | BF863098 | BE238263 |
| BE726095 | BE129400 | BE452421 | BE337587 |
| BE724503 | BE024348 | BF861661 | BE238438 |
| BE122186 | BE452807 | BE024383 | BF862916 |
| BE351871 | BE453047 | BE238286 | BE452223 |
| BE726164 | BF863745 | BE129365 | BE726187 |
| BF861988 | BE024206 | BE452150 | BE452606 |
| BE725215 | BE121701 | BE056787 | BE351649 |
| BE725171 | BE122337 | BE337172 | BE121918 |
| BE726174 | BE725043 | BE726456 | BE725903 |
| BE056399 | BE351680 | BF863412 | BE121753 |
| BE726913 | BF863900 | BE212016 | BE227874 |
| BE237912 | BE351880 | BF864199 | BE452774 |
| BF862637 | BE453590 | BE726495 | BE351714 |
| BE352024 | BF860395 | BF862523 | BF863695 |
| BE761415 | BE211806 | BF863601 | BE129195 |
| BE238167 | BE726867 | BF860377 | BE238351 |
| BE452191 | BE352237 | BE453407 | BF864646 |
| BE122045 | BE121897 | BF862067 | BE212115 |
| BE725780 | BF862890 | BF859730 | BE452503 |
| BE452250 | BE724278 | BE121944 | BE452532 |
| BE238188 | BE724324 | BE726102 | BE237794 |
| BE351782 | BE337155 | BE025168 | BE761188 |
| BE121939 | BF861707 | BE128985 | BE122208 |
| BF861148 | BE211931 | BF862380 | BF860057 |
| BE351772 | BE453115 | BE121633 | BE212358 |
| BE211864 | BE212232 | BF862103 | BE337710 |
| BF866675 | BE452139 | BF860628 | BE122341 |
| BE352316 | BF860968 | BE024889 | BE452149 |
| BE761371 | BE237704 | BE351875 | BE453134 |
| BE122261 | BE337211 | BE122141 | BE122090 |
| BE024622 | BE237970 | BE724397 | BF867375 |
| BE122300 | BF860271 | BE024241 | BE724702 |
| BE452878 | BF865699 | BF859643 | BE726851 |
| BE227301 | BE725731 | BF866463 | BE237705 |
| BE726702 | BF863006 | BF866812 | BE122212 |
| BE351825 | BE122124 | BE024804 | BE725137 |
| BE724687 | BE025133 | BF861449 | BF862789 |
| BF863294 | BE237818 | BE024330 | BF866639 |
| BE121514 | BF862029 | BE211768 | BF862801 |
| BE761382 | BE122394 | BF863677 | BE352288 |
| BE726485 | BE227486 | BE212030 | BE725812 |
| BE726382 | BE452709 | BE724346 | BE227381 |
| BE761296 | BF867755 | BE352192 | BE726542 |
| BF862563 | BE056503 | BE725962 | BE227734 |
| BF863966 | BE237992 | BE056765 | BF863705 |
| BF863829 | BF859866 | BE724739 | BE761292 |
| BE211898 | BE129426 | BF862110 | BE122173 |
| BE724331 | BE337694 | BE761248 | BE351842 |
| BE453412 | BE352070 | BF863308 | BE212253 |
| BF862005 | BF862580 | BE024147 | BE725964 |
| BE452337 | BE725061 | BE129368 | BE122292 |
| BF863256 | BF859855 | BF863354 | BE724641 |
| BE122104 | BE452225 | BE237950 | BF867640 |
| BF860455 | BE024276 | BE122420 | BE453573 |
| BE725849 | BE212158 | BE056617 | BE024670 |
| BE351885 | BE237659 | BE351671 | BE056705 |
| BF867612 | BF864201 | BE351882 | BF865940 |
| BF863380 | BE129157 | BE725207 | BE351986 |
| BF860307 | BE024776 | BF868050 | BE725164 |
| BE056631 | BE352091 | BF859830 | BF862659 |
| BE352226 | BF859667 | BE129208 | BF861910 |
| BE453213 | BE025124 | BF862610 | BE452339 |
| BF866428 | BE337585 | BE352006 | BE024767 |
| BE452624 | BE452239 | BF863254 | BE056703 |
| BE352059 | BF863741 | BE453092 | BE724612 |
| BE122359 | BE211955 | BF863589 | BE024766 |
| BE726992 | BE452887 | BE238070 | BE453600 |
| BE237633 | BE056401 | BF863004 | BE227552 |
| BF864650 | BE725881 | BE352325 | BE211925 |
| BE724936 | BE121459 | BE724953 | BE352248 |
| BE452412 | BE238180 | BE725656 | BE237783 |
| BE351905 | BF862695 | BE725700 | BF863651 |
| BE122415 | BE337176 | BE122232 | BF863310 |
| BF862747 | BE122305 | BF861966 | BE453602 |
| BE352028 | BE238337 | BF859788 | BE351853 |
| BE724269 | BE337213 | BE725314 | BE129458 |
| BE024228 | BE025092 | BE212334 | BE121498 |
| BE121719 | BE024898 | BF863928 | BE129225 |
| BE761184 | BE121987 | BE453130 | BF863847 |
| BE351914 | BF863671 | BE725158 | BF863557 |
| BF862053 | BE726649 | BF863755 | BE121489 |
| BF863691 | BF860868 | BF862418 | BE726219 |
| BE725992 | BE212127 | BF867526 | BE129031 |
| BE227580 | BF863084 | BE453634 | BE725432 |
| BE227454 | BE726904 | BE352188 | BF860183 |
| BE227787 | BF862946 | BF866430 | BE724393 |
| BE025004 | BE725565 | BF864305 | BF862404 |
| BE025019 | BF863130 | BE024562 | BF860552 |
| BE452705 | BE726664 | BF866479 | BE761231 |
| BF863998 | BE024532 | BE726328 | BE122275 |
| BE227419 | BE024611 | BF862547 | BF859690 |
| BF862153 | BE237737 | BE212171 | BE351872 |
| BE725912 | BE129358 | BE452662 | BE351633 |
| BE452580 | BE352169 | BF861713 | BE237681 |
| BE227528 | BE761214 | BE351739 | BE024567 |
| BE238253 | BF859488 | BF863140 | BE352129 |
| BE724573 | BF861912 | BE351814 | BE122166 |
| BF859948 | BF862239 | BE056465 | BE237740 |
| BE761353 | BE122025 | BE726172 | BF861687 |
| BE726708 | BF863874 | BF861822 | BE129311 |
| BE227818 | BE725909 | BE056774 | BE453147 |
| BE725149 | BE725786 | BF860507 | BE453577 |
| BE726349 | BE237862 | BE212129 | BF860099 |
| BF867345 | BE024621 | BE024927 | BE237742 |
| BE337632 | BF865978 | BE024868 | BF860207 |
| BE352121 | BE024189 | BE121547 | BE227641 |
| BE212200 | BF865917 | BE227853 | BE352194 |
| BE228036 | BE352272 | BE122092 | BE056777 |
| BE352196 | BE724823 | BE452572 | BE238002 |
| BE351861 | BE212007 | BE024656 | BE351968 |
| BE129526 | BE337261 | BE725257 | BE725990 |
| BF868271 | BE725002 | BE024890 | BF862791 |
| BE453199 | BE761273 | BE725325 | BE452385 |
| BE351866 | BF860682 | BF860616 | BE453562 |
| BE352016 | BF861629 | BF862453 | BE453626 |
| BF864024 | BE025206 | BE024896 | BE761359 |
| BE211937 | BE725328 | BE211892 | BF862942 |
| BE453156 | BE227725 | BF863641 | BE761472 |
| BE726386 | BF863192 | BF865966 | BE453234 |
| BE726977 | BE024635 | BF860190 | BF866694 |
| BE726408 | BF860864 | BF863418 | BF864634 |
| BE452874 | BE337223 | BF861591 | BE024431 |
| BE453141 | BE351855 | BE726089 | BE237749 |
| BF863922 | BE724839 | BE024624 | BE227797 |
| BF862816 | BE452753 | BF862001 | BF861836 |
| BF860102 | BF862962 | BE726652 | BE024968 |
| BF862588 | BE237964 | BE238111 | BE726143 |
| BF861005 | BE352209 | BE122418 | BF863298 |
| BE337628 | BF864096 | BE452793 | BE337589 |
| BE352123 | BE725145 | BE128972 | BE237864 |
| BE452494 | BF863711 | BE129397 | BF860433 |
| BE352041 | BF865996 | BE725577 | BE211837 |
| BF867113 | BE452227 | BE024265 | BF863954 |
| BE212154 | BE452584 | BE351992 | BE725301 |
| BE238085 | BE121461 | BE724795 | BE452654 |
| BE237631 | BF862469 | BE726931 | BE129131 |
| BE726110 | BE352131 | BF861742 | BE238151 |
| BF860217 | BE122134 | BF866615 | BE724384 |
| BE452131 | BE351808 | BE452557 | BE725716 |
| BE121659 | BE122081 | BE227413 | BE726160 |
| BF860983 | BE452932 | BE128967 | BE024346 |
| BF860740 | BE238370 | BE724904 | BE726318 |
| BE227920 | BF860644 | BE238161 | BE453067 |
| BF860149 | BE453049 | BE227425 | BE453076 |
| BE725429 | BE212226 | BE724281 | BE129239 |
| BE352263 | BE212395 | BE024406 | BF860540 |
| BE211922 | BF864524 | BE211853 | BE024819 |
| BE453402 | BE351895 | BE227503 | BE238039 |
| BF861473 | BE212135 | BE453250 | BE337207 |
| BF864064 | BE725251 | BE724747 | BF862447 |
| BE212272 | BE725985 | BE129007 | BF864569 |
| BE211872 | BE724685 | BE025041 | BF865988 |
| BE237946 | BE724577 | BF859990 | BE024539 |
| BF866804 | BE212005 | BE351691 | BE024523 |
| BE724391 | BE122012 | BE237644 | BE227768 |
| BE761355 | BE452941 | BE726100 | BE024560 |
| BE352220 | BE452896 | BF863687 | BE238428 |
| BF863839 | BE024342 | BE726304 | BE351779 |
| BE122095 | BE726658 | BE024771 | BF866259 |
| BE725953 | BE025099 | BF862120 | BE121631 |
| BF859920 | BE726137 | BE121791 | BE725899 |
| BE725015 | BE121655 | BE724683 | BE024762 |
| BE121679 | BF860640 | BE024810 | BE725839 |
| BE452768 | BE726728 | BE237651 | BE024437 |
| BE724291 | BE352297 | BF862416 | BE724537 |
| BE122067 | BE452578 | BF862531 | BF866157 |
| BE337137 | BE351926 | BE452810 | BE725032 |
| BF860576 | BF860263 | BE237654 | BE452603 |
| BE211971 | BF864614 | BE227411 | BE122138 |
| BF863288 | BF862513 | BE453101 | BE227718 |
| BF863358 | BF866537 | BF861737 | BE453117 |
| BF859659 | BE452373 | BF862623 | BE725071 |
| BE452568 | BF859184 | BF863180 | BF864288 |
| BE024254 | BE453268 | BE024780 | BE337698 |
| BE725870 | BE726077 | BE024841 | BE725265 |
| BE725068 | BE726994 | BE452488 | BE024435 |
| BF859826 | BF859878 | BE024949 | BF861467 |
| BE724255 | BE352203 | BE725247 | BE453150 |
| BE724660 | BE024964 | BE238353 | BE724978 |
| BF863653 | BE726232 | BE024608 | BF863382 |
| BF863547 | BF863986 | BF862715 | BF861597 |
| BE724249 | BE351741 | BE725051 | BF859792 |
| BE352219 | BE212001 | BF863060 | BE724819 |
| BE724636 | BE724731 | BF863733 | BE761318 |
| BE725958 | BE725751 | BE024210 | BF860860 |
| BE724322 | BF863565 | BE726392 | BE238264 |
| BF864070 | BE129534 | BE056691 | BE024652 |
| BE238352 | BF863170 | BF861607 | BE724507 |
| BE726800 | BE352163 | BE238407 | BF863480 |
| BF866684 | BE227478 | BF863496 | BE725951 |
| BE024851 | BE237664 | BE452396 | BF862882 |
| BE352148 | BE237841 | BE122382 | BE761255 |
| BE227770 | BE025056 | BE129107 | BE726631 |
| BE212111 | BE725209 | BE352104 | BF863561 |
| BF860384 | BE237938 | BE726608 | BE237688 |
| BF859579 | BE726741 | BE122101 | BE227543 |
| BF867732 | BE452151 | BF863178 | BF862541 |
| BE337254 | BF862787 | BF863761 | BE352076 |
| BF863124 | BE238313 | BE352097 | BE761525 |
| BE724963 | BF864420 | BF863114 | BF862451 |
| BE761387 | BF862023 | BF859530 | BE056813 |
| BE237637 | BE726918 | BF863300 | BE724720 |
| BF863336 | BE452610 | BF866667 | BF863964 |
| BE725243 | BE025176 | BE351892 | BF862687 |
| BF866696 | BF863152 | BE452746 | BE761284 |
| BE352023 | BE056808 | BE725914 | BE724511 |
| BF862354 | BE761464 | BE024444 | BF860935 |
| BE129441 | BE725217 | BE237881 | BF866066 |
| BE726412 | BE237806 | BE726307 | BE238331 |
| BE024623 | BE725188 | BE761497 | BE121666 |
| BE724535 | BE726967 | BF861463 | BE237883 |
| BE351947 | BE725567 | BF860319 | BE724401 |
| BF863498 | BE726602 | BE352135 | BE352222 |
| BE724891 | BE227467 | BE238377 | BE121559 |
| BF862293 | BE237850 | BE025131 | BF863717 |
| BF861800 | BE452552 | BF861897 | BE227637 |
| BF862013 | BE452679 | BE121766 | BE129309 |
| BE452442 | BE238322 | BE452613 | BE726723 |
| BE337151 | BE025059 | BF863707 | BE351662 |
| BF866915 | BE761227 | BF863727 | BE352179 |
| BE726552 | BE724911 | BE726975 | BE351943 |
| BE238284 | BF867684 | BE121525 | BE761277 |
| BE725495 | BE024605 | BE724832 | BE352175 |
| BE724518 | BF862996 | BE351655 | BE352058 |
| BE724711 | BE056717 | BE351682 | BE351704 |
| BF864773 | BF862410 | BE351801 | BE725955 |
| BE129337 | BE726071 | BE351751 | BE725628 |
| BE453189 | BF863775 | BE352149 | BF865934 |
| BF860406 | BF863402 | BE351769 | BE024614 |
| BF864014 | BF862396 | BE352177 | BE726846 |
| BE025182 | BF864141 | BE352201 | BE024606 |
| BE761233 | BE351851 | BE352141 | BE724889 |
| BE237667 | BE337577 | BE352120 | BE212342 |
| BE452280 | BE724646 | BE352193 | BE024626 |
| BE724434 | BE351749 | BE352173 | BE211838 |
| BE351824 | BE128987 | BE352077 | BE724273 |
| BF867590 | BE237798 | BE724342 | BE725151 |
| BF861537 | BE724883 | BE352184 | BE352242 |
| BE227550 | BE024633 | BE352009 | BE337162 |
| BE452761 | BF860737 | BE352211 | BE725483 |
| BE351868 | BE726502 | BE352278 | BE351738 |
| BE351770 | BE724464 | BE725625 | BE351736 |
| BE724410 | BF862976 | BE725121 | BE227983 |
| BE724791 | BE724938 | BE725563 | BE725536 |
| BF863625 | BM518827 | BE351794 | BE352183 |
| BE725650 | BE724893 | BE352084 | BE352118 |
| BE726996 | BE453447 | BE228017 | BE726671 |
| BF866002 | BF860730 | BM518829 | BE121477 |
| BF867771 | BE724976 | BE351626 | BE122229 |
| BE337648 | BE025189 | BE352152 | BE211973 |
| BE129387 | BF860705 | BE352099 | BE227968 |
| BE453569 | BF860712 | BE724352 | BE227970 |
| BE725729 | BE024659 | BE351723 | BE228025 |
| BF865976 | BF860841 | BE352138 | BE238334 |
| BE725820 | BE227610 | BE227964 | BE351699 |
| BE452261 | BF863232 | BE351702 | BE351742 |
| BE351955 | BE724875 | BE351663 | BE351805 |
| BE724672 | BE237975 | BE725427 | BE351967 |
| BE129381 | BE227937 | BE352114 | BE351970 |
| BE725260 | BF866889 | BE352195 | BE351985 |
| BF859758 | BF861483 | BE227974 | BE352000 |
| BE724630 | BF860705 | BE352259 | BE352056 |
| BE351829 | BE453558 | BE352320 | BE352087 |
| BE724920 | BE211974 | BE351939 | BE352156 |
| BE725758 | BE227951 | BE352134 | BE352207 |
| BE351963 | BE351990 | BE725594 | BE352230 |
| BE238426 | BE352101 | BE351729 | BE352261 |
| BE024268 | BE351806 | BE352250 | BE352273 |
| BF866661 | BF860584 | BE351731 | BE352285 |
| BE212217 | BE351622 | BE352317 | BE352290 |
| BF859219 | BE351696 | BE725516 | BE352298 |
| BE724270 | BE351789 | BE351812 | BE352336 |
| BE724319 | | | |
| BE725556 | | | |
| BF859160 | | | |
| BF860010 | | | |
| BF860572 | | | |
| BF860795 | | | |
| BF860795 | | | |
| BF861485 | | | |
| BF862205 | | | |
| BF862665 | | | |
| BF867548 | | | |
| BE056562 | | | |

<160> NUMBER OF SEQ ID NOS: 9
<210> SEQ ID NO 1
<211> LENGTH: 17
<212> TYPE: PRT
<213> ORGANISM: Chlamydomonas reinhardtii
<400> SEQUENCE: 1
Val Leu Phe Gly Thr Thr Gly Gly Val Met Gl
#u Ala Ala Leu Arg Thr
1 5
# 10
# 15
Ala
<210> SEQ ID NO 2
<211> LENGTH: 17
<212> TYPE: PRT
<213> ORGANISM: Scenedesmus obliquus
<400> SEQUENCE: 2
Val Leu Phe Gly Thr Thr Gly Gly Val Met Gl
#u Ala Ala Leu Arg Thr
1 5
# 10
# 15
Val
<210> SEQ ID NO 3
<211> LENGTH: 17
<212> TYPE: PRT
<213> ORGANISM: Megasphaera elsedenii
<400> SEQUENCE: 3
Arg Ile Phe Gly Asn Ser Gly Gly Val Met Gl
#u Ala Ala Ile Arg Thr
1 5
# 10
# 15
Ala
<210> SEQ ID NO 4
<211> LENGTH: 17
<212> TYPE: PRT
<213> ORGANISM: Desulfovibrio desulfuricans
<400> SEQUENCE: 4
Thr Ile Phe Gly Val Thr Gly Gly Val Met Gl
#u Ala Ala Leu Arg Phe
1 5
# 10
# 15
Ala
<210> SEQ ID NO 5
<211> LENGTH: 17
<212> TYPE: PRT
<213> ORGANISM: Clostridium pasteurianum
<400> SEQUENCE: 5
Ala Ile Phe Gly Ala Thr Gly Gly Val Met Gl
#u Ala Ala Leu Arg Ser
1 5
# 10
# 15
Ala
<210> SEQ ID NO 6
<211> LENGTH: 17
<212> TYPE: PRT
<213> ORGANISM: Nyctotherus ovalis
<400> SEQUENCE: 6
Asn Leu Phe Gly Val Thr Gly Gly Val Met Gl
#u Ala Ala Ile Arg Thr
1 5
# 10
# 15
Ala
<210> SEQ ID NO 7
<211> LENGTH: 7
<212> TYPE: PRT
<213> ORGANISM: Chlamydomonas reinhardtii
<400> SEQUENCE: 7
Gly Gly Val Met Glu Ala Ala
1 5
<210> SEQ ID NO 8
<211> LENGTH: 21
<212> TYPE: DNA
<213> ORGANISM: artificial sequence
<220> FEATURE:
<223> OTHER INFORMATION: synthetic construct
<400> SEQUENCE: 8
ggyggygtsa tggaggcbgc b
#
#
#21
<210> SEQ ID NO 9
<211> LENGTH: 21
<212> TYPE: DNA
<213> ORGANISM: artificial sequence
<220> FEATURE:
<223> OTHER INFORMATION: synthetic constuct
<400> SEQUENCE: 9
bcgbcggagg tastgyggyg g
#
#
#21

1. A method comprising:
(a) culturing two or more genomically diverse microorganisms under conditions in which at least two genomically diverse microorganisms perform a desired function;
(b) measuring the level of performance by the at least two genomically diverse microorganisms of the desired function;
(c) isolating mRNA from the at least two genomically diverse microorganisms that perform the desired function at different levels;
(d) hybridizing the mRNA or a nucleic acid derivative thereof to a microarray containing one or more immobilized cDNA sequences; and
(e) identifying one or more opportune targets that are expressed at a higher level in a microorganism that performs the desired function at a higher level compared to the expression level of the opportune target in a different microorganism that performs the desired function at a lower level.

2. The method of claim 1, further comprising:
(f) expressing the one or more opportune targets in a transformed test strain in operable linkage with a heterologous promoter other than the natural promoter(s) of the one or more opportune targets; and
(g) screening or selecting for an increase in the level of performance of the desired function in the transformed test strain compared to a nontransformed test strain.

3. The method of claim 2, further comprising identifying a transformed test strain that exhibits an increase in the desired function compared to the nontransformed test strain.

4. The method of claim 3 wherein at least two independent transformed test strains expressing different opportune targets are identified.

5. The method of claim 4 wherein:
(a) the at least two independent transformed test strains are placed in conditions where they undergo nucleic acid exchange; and
(b) progeny cells from the nucleic acid exchange are screened or selected for a further increase in the desired function at a level higher than that exhibited by at least one of the at least two independent transformed test strains.

6. The method of claim 5, wherein the progeny cells from the nucleic acid exchange are screened or selected for a further increase in the desired function at a level higher than that exhibited by all of the at least two independent transformed test strains.

7. The method of claim 6, wherein:
(a) a first progeny cell that exhibits a further increase in the desired function at a level higher than that exhibited by all of the at least two independent transformed test strains is placed in conditions where it undergoes nucleic acid exchange with a second distinct progeny cell that also exhibits a further increase in the desired function at a level higher than that exhibited by all of the at least two independent transformed test strains to produce additional progeny; and
(b) screening or selecting the additional progeny for performance of the desired function at a level higher than that exhibited by at least one of the first or second progeny cells.

8. The method of claim 7, wherein the additional progeny are screened or selected for performance of the desired function at a level higher than that exhibited by the first and second progeny cells.

9. The method of claim 1, wherein the desired function is selected from the group consisting of hydrogen production, carbon sequestration, astaxanthin production, dissolved solid transport, transport of Na⁺ of a sodium salt, transport of Cl⁻ of a salt containing chlorine, and degradation or chelation of an environmental toxin.

10. The method of claim 9, wherein the desired function is hydrogen production, and the desired function is screened using a multiwell plate of independent genomically diverse microorganisms in liquid culture media, and an increase in hydrogen production is identified by a change in optical properties of a chemochromic film placed on top of the plate.

11. The method of claim 1, wherein at least one of the two or more genomically diverse microorganisms is listed in Tables 1, 2 or 3.

12. The method of claim 1, wherein the two or more genomically diverse microorganisms are generated by inducing genomic diversity through mutagenesis of cells.

13. The method of claim 1, wherein a plurality of distinct microarrays are used, each microarray containing nucleic acid sequences that encode the same set of protein sequences but wherein at least two distinct microarrays from the plurality encode the protein sequences using different codon usage regimes.

14. The method according to claim 5, wherein the nucleic acid exchange is selected from the group consisting of sexual recombination, bacterial conjugation, virus-mediated nucleic acid exchange, and protoplast fusion.

15. The method according to claim 14, wherein the at least two independent transformed test strains are green algae and the sexual recombination is induced by removing nitrogen from the culture media.

16. The method according to claim 1, wherein distinct culture conditions are used to induce the genomically diverse microorganisms to perform the same desired function.

17. The method of claim 5, wherein the same heterologous promoter drives expression of all opportune targets.

18. The method of claim 1, wherein at least 40 genomically diverse independent strains of microorganisms of a species are analyzed.

19. The method of claim 1, wherein at least 2 genomically diverse independent strains of microorganisms from each of at least 2 distinct species are analyzed.

20. A microarray containing a plurality of immobilized nucleic acid sequences, wherein the nucleic acid sequences encode protein sequences using preferred codons of a species other than the species from which the protein sequences are obtained.

21. The microarray of claim 20, wherein the nucleic acid sequences encode protein sequences using most preferred codons of a species other than the species from which the protein sequences are obtained.
